(19) United States
(12) Patent Application Publication
(10) Pub. No.: US 2011/0268800 A1
(43) Pub. Date: Nov. 3, 2011

(54) Unsaturated fatty acids as thrombin inhibitors

(75) Inventor: Hans-Rainer Hoffmann, Neuwied (DE)

(73) Assignee: LTS Lohmann Therapie-Systeme AG, Andernach (DE)

(21) Appl. No.: 13/134,938

(22) Filed: Jun. 21, 2011

Related U.S. Application Data
(62) Division of application No. 12/084,438, filed on May 1, 2008.

(30) Foreign Application Priority Data
Dec. 6, 2005 (DE) 10 2005 058 369.5

(51) Int. Cl. A61K 31/20; A61P 7/02; C11C 1/04; A61K 9/28; C11C 3/00

(52) U.S. Cl. 424/474; 514/560; 554/223; 554/219

(57) ABSTRACT

A preparation having at least one unsaturated fatty acid and the use of at least one unsaturated fatty acid having a chain length of 18 or 20 carbon atoms and of plant drugs containing the unsaturated fatty acid as a free fatty acid or a fatty acid radical of a triglyceride. The fatty acid has a chain length of 18 carbon atoms which are provided with 1 to 3 double bonds and the fatty acid has a chain length of 20 carbon atoms which are provided with 1 to 4 double bonds. The double bond or one of the double bonds are located in position 9 or 11 of the carbon chain. The unsaturated fatty acid is supplied in the all-cis configuration. The at least one unsaturated fatty acid prevents and/or treats or is used for producing a preparation for preventing and/or treating thrombosis and thromboembolic diseases.

### CROSS-REFERENCE TO RELATED APPLICATIONS

This application is a divisional application of co-pending U.S. application Ser. No. 12/084,438 filed on May 1, 2008, which is a National Stage application of International Application No. PCT/EP2006/011497, filed on Nov. 30, 2006, which claims priority of German application number 10 2005 058 369.5, filed on Dec. 6, 2005, all of which are incorporated herein by reference in their entireties.

### BACKGROUND OF THE INVENTION

1. Field of the Invention

The present invention relates to a preparation having at least one unsaturated fatty acid and the use of at least one unsaturated fatty acid. More particularly, the present invention relates to a preparation having at least one unsaturated fatty acid and the use of at least one unsaturated fatty acid for preventing and/or treating thrombosis and thromboembolic diseases.

2. Description of the Prior Art

Thrombosis is defined as intravital blood coagulation inside a vessel or a cardiac cavity caused by a fibrin-containing platelet aggregate, called thrombus. Hence, thrombosis is the formation of a blood clot in the path of the blood flow which does not serve to effect haemostasis following injury but obstructs the flow of blood in the vessel concerned. If the thrombus dislodges and is carried to narrowing vessels, with the consequence of those vessels being occluded, this is called thromboembolism.

Thrombosis is the cause or contributes to half of the overall number of deaths in Germany. More than eighty percent of these deaths occur within the group of those over 60 years of age. According to estimates, this age group will account for 37% of the population in the year 2050. Already from the year 2020, the group of 50- to 64-year-olds will provide 39% of the potential labour force.

Anticoagulants and platelet aggregation inhibitors are available for the primary and secondary prophylaxis of thromboembolic disorders. Within the 33 indication groups with the highest number of prescriptions in 2003, anticoagulants showed the highest rate of increase (+11.8%), and platelet aggregation inhibitors the third-highest rate of increase (+10.5%). Between 1994 and 2003, the number of prescriptions of anticoagulants almost tripled and the number of prescriptions of platelet aggregation inhibitors almost sextupled. The market for antithrombotic medicaments is expected to more than double within the period from 2001 to 2010.

In Germany alone, about half a million people are currently being treated with anticoagulants. Heparins and vitamin K antagonists have been utilised with success in the therapy of thromboembolic disorders for over 50 years. Nevertheless, there are limitations to the use of each of the two substance groups. Heparins must be applied parenterally and inhibit only the free thrombin, not the thrombin which is bound to fibrin. Vitamin K antagonists have a narrow therapeutic window while at the same time involving a high risk of interaction with nourishments and medicaments, as well as having interindividually variable kinetics. For these reasons, an insufficient number of people are being treated with anticoagulants despite the need for such treatment. Thus, 41% of all patients suffering from atrial fibrillation do not receive antithrombotic therapy, although their risk of apoplexia is five times as high.

In June 2004, Ximelagatran was approved for indications of short-term prophylaxis of thromboembolism throughout Europe (with the exception of Great Britain and Ireland) as the first direct thrombin inhibitor which is orally available. This active substance meets many demands made on an ideal anticoagulant. It can be administered in a fixed dose; it exhibits a quick onset of action and inhibits also fibrin-bound thrombin. The efficacy of this active substance has been proved in numerous studies. However, two American studies (EXULT A; EXULT B) have shown that liver damage may occur at higher dosage as well as in longer-term application. For this reason, the US Food and Drug Administration, FDA, in October 2004, refused approval of this active substance. With patients suffering from liver dysfunction, Ximelagatran is contraindicated. The Federal Ministry for Drugs and Medicinal Products recommends monitoring the liver function 4-6 weeks after termination of the therapy. Thus, it is presently questionable whether Ximelagatran will prove to be of value in long-term therapy, and thereby as a replacement for vitamin K antagonists.

The disadvantages of the known anticoagulants and platelet aggregation inhibitors show that there is a need for new preparations for the prophylaxis and treatment of thromboses and thromboembolism which do not have these disadvantages.

### SUMMARY OF THE PRESENT INVENTION

The object of this invention was therefore to provide new active substances and preparations for the prophylaxis and/or treatment of thromboses and thromboembolisms.

This object has been achieved by identifying unsaturated fatty acids as inhibitors of thrombin, wherein said unsaturated fatty acids have a chain length of 18 or 20 carbon atoms, the unsaturated fatty acids having a chain length of 18 carbon atoms containing 1 to 3 double bonds in their carbon chain and the unsaturated fatty acids having a chain length of 20 carbon atoms containing 1 to 4 double bonds in their carbon chain, the double bond or one of the double bonds being located at position 9 or 11 of the carbon chain, and the unsaturated fatty acids being present in the all-cis configuration.

It is astounding that in Germany the death rate due to cardiovascular diseases is higher than in the Mediterranean countries, although in the latter alcohol and nicotine abuse are considerably more wide-spread than in Germany. For this phenomenon, the technical term “Mediterranean paradox” is to be found in medical literature as well as in the press.

Since to date no genetic factors could be identified as the cause of the “Mediterranean paradox”, the reason for this phenomenon is being sought in the difference in people's diets. The more frequent and quantitatively higher consumption of fresh salad in the Mediterranean is particularly striking in this connection. Presumably, there are plant ingredients which are able to guard against cardiovascular problems. Extending the assumption further, the inventors assumed that these ingredients have an influence on the coagulability of the blood so that infarct formation is prevented. One possibility would be that some of the ingredients act as inhibitors of thrombin.

To verify this assumption, dichloromethane and methanol extracts were made from the plant drugs of 78 plants and were tested for thrombin inhibition.

As the testing system, an amidolytic assay with Chromozym TH (tosylglycyl-prolyl-arginine-4-nitroanilide acetate) was used as a substrate for thrombin. The principle of the test system is based on the cleavage of the substrate by thrombin, and on the release of p-nitroaniline, which absorbs at a wavelength of 405 nm. The absorption increase over time was detected photometrically. The inhibition of thrombin was calculated by a comparison between the linear initial phase of the absorption increase of the test samples containing the extract, and the blank value.

After precipitating and withdrawing the tanning agents contained in the extracts by adding a polyvinyl pyrrolidone solution to the extracts and subsequent centrifugation, the methanol extracts of Fructus Cardamomi, Fructus Sabalae, Semen Erucae, Fructus Anisi and Fructus Foeniculi, as well as the dichloromethane extracts of Flores Sambuci, Herba Adonidis, Radix Althaeae, Fructus Anisi, Fructus Carvi, Fructus Coriandri and Fructus Sabalae were found to cause a 50% inhibition of thrombin. With these plant drugs, removal of the tanning agents resulted, at best, in an irrelevant reduction of the thrombin-inhibiting effect of the extracts.

In the above investigation, the extracts of three of these plant drugs were found to inhibit thrombin particularly effectively: Semen Erucae, Flores Sambuci and Herba Adonidis.

Semen Erucae is the seeds of white mustard (Sinapis alba L.). White mustard belongs to the family of Brassicaceae. This annual plant is 20-60 cm high and bears a 2-to-4-cm-long pod as fruit; this pod contains the brownish to whitish seeds. The plant is cultivated in northern America as well as in northern and western Europe, but also occurs in Siberia and eastern Asia. The seeds are used as the drug (Semen Erucae). The drug contains glucosinolates. The main glucosinolate is sinalbin, from which, in the presence of water, the non-volatile sinalbin mustard oil p-hydroxybenzyl mustard oil is produced by the enzyme myrosinase. Apart from this, Semen Erucae contains 20-35% fatty oils having a high content of unsaturated fatty acids (above all, eruca acid), proteins (approx. 40%) and phenyl propane derivatives (above all, sinapine). Semen Erucae has an antibacterial, skin-irritating and hyperaemia-inducing effect. Semen Erucae is used externally in cases of catarrh of the respiratory tracts, soft tissue rheumatism as well as chronic degenerative arthropathy.

As Flores Sambuci, the dried flowers of the elder (Sambucus nigra L.) are used, which belongs to the family of Caprifoliaceae. This plant is a shrub or tree which is up to 7 m high. The yellowish-white flowers form cymes with 5 main stalks. Flores Sambuci contains up to 3% flavonoids, essential oils, sterols and triterpenes. An infusion of elderflowers leads to increased bronchial secretion and is used in cases of catarrh of the respiratory tracts as well as dry cough. Furthermore, the diaphoretic action of Flores Sambuci is used in popular medicine for the treatment of feverish colds.

Herba Adonidis is the foliage of the pheasant's eye (Adonis vernalis L.), which belongs to the family of the Ranunculaceae. This plant is 10-40 cm high and is primarily cultivated in Bulgaria, Russia and Hungary. The foliage is collected in the bloom period. Herba Adonidis contains 0.2-0.8% cardioactive glycosides of the cardenolide type, mainly adonitoxigenin glycosides and strophantidin glycosides (e.g. cymarin), as well as 1% flavonoids with the main component adonivernith. The drug has a positive inotropic effect as well as a venoconstrictive effect, and is used in cases of cardiac insufficiency (NYHA grade I-II), particularly cardiac insufficiency with concomitant nervous symptoms.

After screening, the above three drugs, which inhibit thrombin particularly effectively, were further examined. Among other things, all of the lipids contained in these drugs were extracted with a mixture of hexane and isopropanol (3:2), the free fatty acids were individually separated by preparative thin layer chromatography and identified by means of mass spectrometry. In each of the three drugs, fatty acids were surprisingly identified as the thrombin-inhibiting principle. In Herba Adonidis, linoleic acid and linolenic acid are responsible for the thrombin-inhibiting action. However, palmitic acid, which likewise occurs in this drug, was without effect. In the two remaining drugs, oleic acid and eicosenoic acid were identified as the thrombin-inhibiting substances apart from linoleic and linolenic acid.

After identifying the fatty acids as inhibitors of thrombin, a number of fatty acids were examined for thrombin-inhibiting action. The results of this investigation are summarised in Table 1.

TABLE 1

| | | Number of carbon | Systematic name |
|---|---|---|---|
| | Inhibition | atoms:Number of | of the fatty |
| Trivial name | (%) | double bonds | acids |
| 9-Decenoic acid | −3.7 | 10:1 | 9-Decenoic acid |
| Docosahexaenoic | −2.5 | 20:6 | 4-cis, 7-cis, 10-cis, |
| acid | | | 13-cis, 16-cis, 19-cis- |
| | | | Docosahexaenoic acid |
| Eicosapentaenoic | −1.0 | 20:5 | 5-cis, 8-cis, 11-cis, |
| acid | | | 14-cis, 17-cis-Eicosa- |
| | | | pentaenoic acid |
| Octanoic acid | 0.7 | 8:0 | Octanoic acid |
| Palmitic acid | 1.4 | 16:0 | Hexadecanoic acid |
| Stearidonic acid | 2.4 | 18:4 | 6-cis, 9-cis, 12-cis, |
| | | | 15-cis-Octadeca- |
| | | | tetraenoic acid |
| Elaidinic acid | 7.4 | 18:1 | 9-trans-Octadecenoic |
| | | | acid |
| Eruca acid | 7.8 | 22:1 | 13-cis-Docosaenoic acid |
| Ricinoleic acid | 43.9 | 18:1 | 12-Hydroxy-9-cis- |
| | | | octadecenoic acid |
| Eicosenoic acid | 60.7 | 20:1 | 11-cis-Eicosenoic acid |
| Oleic acid | 65.8 | 18:1 | 9-cis-Octadecenoic acid |
| Linoleic acid | 71.7 | 18:2 | 9-cis,12-cis-Octadeca- |
| | | | dienoic acid |
| Arachidonic acid | 75.2 | 20:4 | 5-cis, 8-cis, 11-cis, |
| | | | 14-cis-Eicosa- |
| | | | tetraenoic acid |
| Linolenic acid | 75.5 | 18:3 | 9-cis, 12-cis, 15-cis- |
| | | | Octadecatrienoic acid |
| Eicosatrienoic | 76.0 | 20:3 | 11-cis, 14-cis, 17-cis- |
| acid | | | Eicosatrienoic acid |
| Vaccenic acid | 79.9 | 18:1 | 11-cis-Octadecenoic |
| | | | acid |

The front-runner, with 80% inhibition, is cis-vaccenic acid, which occurs, for example, in cereals and soya, but also in sea-buckthorn, radish, lilium, burdock, lotus and carrot; followed by eicosatrienoic acid, linolenic acid, arachidonic acid, linoleic acid and oleic acid, which still show a 66% inhibition of thrombin.

The thrombin-inhibiting fatty acids are unsaturated fatty acids which are characterised by the following features:
a carbon chain of 18 or 20 carbon atoms;
1 to 3 carbon-carbon double bonds in the carbon chain if the fatty acid has a chain length of 18 carbon atoms, or
1 to 4 carbon-carbon double bonds in the carbon chain if the fatty acid has a chain length of 20 carbon atoms;
the double bond or one of the double bonds is located at position 9 or 11 in the carbon chain; and
the unsaturated fatty acids are present in the all-cis configuration.

For the thrombin-inhibiting action, it is irrelevant whether the fatty acid is of natural or synthetic origin.

While the favourable influence of certain fatty acids on various parameters of cardiovascular diseases is known, the thrombin inhibition found here has so far not been described and is likely to be an essential component of the action at the basis of the “Mediterranean paradox.” Oleic acid, linoleic acid and linolenic acid are contained in edible oils (e.g. olive oil) used for preparing salads. They are supplied to the body in appropriate doses via the lymph system, almost like a continuous infusion.

It has heretofore been unknown that certain unsaturated fatty acids inhibit thrombin. Hence, a non-toxic class of compounds enters into the focus of the cardiovascular specialists, making it possible to provide new preparations for the prophylaxis and/or treatment of thromboses and thromboembolic diseases that should not have the disadvantages of the known anticoagulants and platelet aggregation inhibitors.

### DETAILED DESCRIPTION OF THE PRESENT INVENTION

The subject matter of the present invention is therefore the use of one or more unsaturated fatty acids, the carbon chain of which has 18 or 20 carbon atoms—the unsaturated fatty acids having a chain length of 18 carbon atoms containing 1 to 3 double bonds in their carbon chain and the unsaturated fatty acids having a chain length of 20 carbon atoms containing 1 to 4 double bonds in their carbon chain, the double bond or one of the double bonds being located at position 9 or 11 of the carbon chain, and the unsaturated fatty acids being present in the all-cis configuration—for the prophylaxis and/or treatment and for the production of a preparation for the prophylaxis and/or treatment of thrombosis and thromboembolic diseases.

With particular preference, the fatty acid or the fatty acids is/are selected from the group of fatty acids comprising oleic acid, linoleic acid, linolenic acid, eicosenoic acid, ricinoleic acid, vaccenic acid, eicosatrienoic acid and arachidonic acid.

The present invention furthermore relates to the use of plant drugs which contain at least one of the thrombin-inhibiting fatty acids, for the prophylaxis and/or treatment of thromboses and thromboembolic diseases, and for the production of a preparation for the prophylaxis and/or treatment of thromboses and thromboembolic diseases. The fatty acid may either be present as a free fatty acid in the plant drug, or as a fatty acid residue of a triglyceride in the fat or oil contained in the plant drug, as, for the most part, the fatty acids are present in the plants as triglycerides, from which they are derived by hydrolysis. However, the fatty acids are resynthesised with monoacyl glycerols to triglycerides in the intestinal mucosa; they are then transported away as chylomicrons by the lymph, and bound to lipoproteins in the serum.

Hence, the present invention also relates to the use of plant drugs containing a fat or oil wherein at least one thrombin-inhibiting fatty acid is present as a fatty acid residue of one of the triglycerides contained in the fat or oil, for the prophylaxis and/or treatment of thromboses and thromboembolic diseases, and for the production of a preparation for the prophylaxis and/or treatment of thromboses and thromboembolic diseases.

The plant drug preferably is Fructus Cardamomi, Fructus Sabalae, Fructus Anisi, Fructus Foeniculi, Radix Althaeae, Fructus Carvi, Fructus Coriandri or Fructus Sabalae, especially preferably Semen Erucae, Flores Sambuci or Herba Adonidis.

The present invention also relates to preparations for the prophylaxis and/or treatment of thromboses and thromboembolic diseases which contain as active substance at least one unsaturated fatty acid having a carbon chain with 18 or 20 carbon atoms—the fatty acid with a chain length of 18 carbon atoms having 1 to 3 double bonds in its carbon chain and the fatty acid with a chain length of 20 carbon atoms having 1 to 4 double bonds in its carbon chain, the double bond or one of the double bonds being located at position 9 or 11 of the carbon chain, and the unsaturated fatty acid being present in the all-cis configuration—or which contain at least one plant drug containing at least one of these fatty acids, or a triglyceride with at least one of these fatty acids, as fatty acid residue.

Preferably, the unsaturated fatty acid in said preparation for the prophylaxis and/or treatment of thromboses and thromboembolic diseases is selected from the group consisting of oleic acid, linoleic acid, linolenic acid, eicosenoic acid, ricinoleic acid, vaccenic acid, eicosatrienoic acid and arachidonic acid.

The preparation according to the present invention may, for instance, be a medicament or a food supplement. However, it is also possible to supply the preparation as a dietetic salad oil or, for example seasoned, as an additive for soups or sauces.

The preparation according to the invention may be present in solid as well as in liquid or semi-liquid form. Suitable forms for administering the preparations according to the invention are, for example, powders, granules, capsules, tablets, coated tablets, suspensions, solutions, emulsions, ointments, gels, creams or pastes. The basic materials and auxiliary substances which are commonly used for the production of the respective administration forms are in principle known to those skilled in the art.

Preferably, the preparations according to the present invention are preparations to be administered orally, but preparations present in a parenterally administrable form are also suitable, for example an injection solution on an anhydrous basis for intramuscular or subcutaneous administration.

It is particularly preferred that the preparation according to the present invention be present in a form that is to be administered transdermally, for example as a transdermal therapeutic system (pressure-sensitive adhesive active substance patch).

In a particularly preferred embodiment, the preparation according to the invention has depot action, which means that after application it delivers the thrombin-inhibiting fatty acid(s) continuously, for a prolonged period of time, to the organism of the patient to be treated. Suitable preparations having depot action are the above-mentioned injection solutions on anhydrous basis, and transdermal therapeutic systems. Further suitable administration forms are gastroretentive systems for oral application. The base materials and auxiliary substances commonly used for producing preparations with a depot effect are also known to those skilled in the art, as are the methods that may be applied for that purpose.

### Example

For the thrombin inhibition test, the method according to Sturzebecher was employed, with the exception of the enzyme concentration (Sturzebecher, J., Vieweg, H. (1995). Structure-Activity Relationships of Inhibitors derived from 3-Amidinophenyl-alanine. J Enzyme Inhib, 9, 87-99).
Buffer: 608 mg Tris buffer and 900 mg NaCl were dissolved in 100 ml bidistilled water. The pH value of the buffer was subsequently set to 8.0, using HCl (2 mMol) (concentrations in the sample: 0.05 mol/1 Tris HCL, 0.154 mol/1 NaCl). The substance to be examined was dissolved in ethanol (5% total volume). This was followed by filling up with buffer.
Enzyme: 1 mg/ml (=50 NIH—U/ml) thrombin was dissolved in 0.9% NaCl solution (stock solution). To prepare the sample, this solution was diluted 1:100 with a solution of 0.9% NaCL and 1% bovine serum albumin.
Substrate: 6.6 mg Chromozym TH was dissolved in 10 ml bidistilled water (concentration=1 mmol/l).
Measurement sample: 200 μl buffer/substance
   25 μl substrate
   50 μl enzyme
Conditions: Room temperature
Implementation: Buffer and enzyme are added together by pipetting and incubated for 15 min. The reaction was started by adding the substrate.
   For measuring the absorption, a plate reader, iEMS Reader MF from Labsystems, was used with 96-well microtitre plates. The measurements took place at a wavelength of 405 nm, every 10 seconds, 60 measurements. Prior to start of the measurements, the microtitre plate was shaken at 600 rpm.
Evaluation: The measurements were recorded by means of iEMS Accent Software, and evaluated with Microsoft Excel 97 Software.
   To calculate inhibition, the slope in the linear initial phase of the absorption increase in the samples containing the substance to be examined was compared with the slope of the absorption increase in the blank sample. For evaluation, the first 5 measured values were left out of consideration since the bubbles forming on account of the mixing can lead to problems at the beginning of the reaction. The inhibition is calculated as follows:

Inhibition=100−(slope_{Inhibitor}/slope_{Blank value}*100)

The correlation between time and absorption was calculated with the “CORREL” function of Excel for the same section.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

I claim:

1. A method for inhibiting thrombin, wherein said method comprises the step of applying to an individual a thrombin-inhibiting preparation containing at least one unsaturated fatty acid as an active ingredient, said at least one unsaturated fatty acid comprising a carbon chain length of 18 or 20 carbon atoms and, if said at least one unsaturated fatty acid has a chain length of 18 carbon atoms said at least one unsaturated fatty acid contains 1 to 3 double bonds in said carbon chain, and if said at least one unsaturated fatty acid has a chain length of 20 carbon atoms said at least one unsaturated fatty acid contains 1 to 4 double bonds in carbon chain, and wherein the double bond or one of the double bonds is located at position 9 or 11 of the carbon chain, and wherein said at least one unsaturated fatty acid is present in an all-cis configuration.

2. The method according to claim 1, wherein said at least one unsaturated fatty acid, or one of said at least one unsaturated fatty acid, is selected from the group consisting of oleic acid, linoleic acid, linolenic acid, eicosenoic acid, ricinoleic acid, vaccenic acid, eicosatrienoic acid and arachidonic acid.

3. The method according to claim 1, wherein said thrombin-inhibiting preparation is applied in a form selected from the group consisting of a medicament, a food supplement, a dietetic salad oil, an additive for soups and an additive for sauces.

4. The method according to claim 1, wherein said thrombin-inhibiting preparation is applied in a form selected from the group consisting of liquid form, semi-solid form, and solid form.

5. The method according to claim 1, wherein said thrombin-inhibiting preparation is applied in a form selected from the group consisting of powders, granules, capsules, tablets, coated tablets, suspensions, solutions, emulsions, ointments, gels, creams and pastes.

6. The method according to claim 1, wherein said thrombin-inhibiting preparation is applied in an orally administrable form.

7. The method according to claim 1, wherein said thrombin-inhibiting preparation is applied in a parenterally administrable form.

8. The method according to claim 7, wherein said thrombin-inhibiting preparation is applied in a transdermally administrable form.

9. The method according to claim 1, wherein said thrombin-inhibiting preparation that is applied has depot action.

10. A method for inhibiting thrombin, wherein said method comprises the step of applying to an individual a thrombin-inhibiting preparation containing at least one plant drug as an active ingredient, wherein said at least one plant drug contains at least one unsaturated fatty acid or at least one triglyceride with said at least one unsaturated fatty acid, or with at least one of said at least one unsaturated fatty acid as fatty acid residue, said at least one unsaturated fatty acid having a carbon chain with a chain length of 18 or 20 carbon atoms and, in the case of a chain length of 18 carbon atoms, containing 1 to 3 double bonds in said carbon chain and, in the case of a chain length of 20 carbon atoms, containing 1 to 4 double bonds in said carbon chain, and wherein the double bond or one of the double bonds is located at position 9 or 11 of the carbon chain, and wherein said at least one unsaturated fatty acid is present in an all-cis configuration.

11. The method according to claim 10, wherein said at least one plant drug is selected from the group of plant drugs consisting of Fructus Cardamomi, Fructus Sabalae, Fructus Anisi, Fructus Foeniculi, Radix Althaeae, Fructus Carvi, Fructus Coriandri, Fructus Sabalae, Semen Erucae, Flores Sambuci and Herba Adonidis.

12. The method according to claim 10, wherein said thrombin-inhibiting preparation is applied in a form selected from the group consisting of a medicament, a food supplement, a dietetic salad oil, an additive for soups and an additive for sauces.

13. The method according to claim 10, wherein said thrombin-inhibiting preparation is applied in a form selected from the group consisting of liquid form, semi-solid form, and solid form.

14. The method according to claim 10, wherein said thrombin-inhibiting preparation is applied in a form selected from the group consisting of powders, granules, capsules, tablets, coated tablets, suspensions, solutions, emulsions, ointments, gels, creams and pastes.

15. The method according to claim 10, wherein said thrombin-inhibiting preparation is applied in an orally administrable form.

16. The method according to claim 10, wherein said thrombin-inhibiting preparation is applied in a parenterally administrable form.

17. The method according to claim 16, wherein said thrombin-inhibiting preparation is applied in a transdermally administrable form.

18. The method according to claim 10, wherein said thrombin-inhibiting preparation that is applied has depot action.

19. A method for producing a preparation for inhibiting thrombin, wherein said method comprises the step of preparing said preparation having at least one unsaturated fatty acid, wherein said at least one unsaturated fatty acid has a carbon chain with a chain length of 18 or 20 carbon atoms, and wherein if the unsaturated fatty acid has a chain length of 18 carbon atoms, said at least one unsaturated fatty acid contains 1 to 3 double bonds in said carbon chain, and wherein if said at least one unsaturated fatty acid has a chain length of 20 carbon atoms, said at least one unsaturated fatty acid contains 1 to 4 double bonds in said carbon chain, and wherein the double bond or one of the double bonds is located at position 9 or 11 of the carbon chain, and wherein the unsaturated fatty acid is present in an all-cis configuration.

20. The method according to claim 19, wherein said at least one unsaturated fatty acid is selected from the group consisting of oleic acid, linoleic acid, linolenic acid, eicosenoic acid, ricinoleic acid, vaccenic acid, eicosatrienoic acid and arachidonic acid.

21. The method according to claim 19, wherein the preparation is present in a form selected from the group consisting of a medicament, a food supplement, a dietetic salad oil, an additive for soups and an additive for sauces.

22. A method for producing a preparation for inhibiting thrombin, wherein said method comprises the step of preparing said preparation having at least one plant drug, wherein said at least one plant drug contains at least one unsaturated fatty acid or at least one triglyceride with said at least one unsaturated fatty acid, or with at least one of said at least one unsaturated fatty acid as fatty acid residue, said at least one unsaturated fatty acid having a carbon chain with a chain length of 18 or 20 carbon atoms and, in the case of a chain length of 18 carbon atoms, containing 1 to 3 double bonds in said carbon chain and, in the case of a chain length of 20 carbon atoms, containing 1 to 4 double bonds in said carbon chain, and wherein the double bond or one of the double bonds is located at position 9 or 11 of the carbon chain, and wherein said at least one unsaturated fatty acid is present in an all-cis configuration.

23. The method according to claim 22, wherein said at least one plant drug is selected from the group of plant drugs consisting of Fructus Cardamomi, Fructus Sabalae, Fructus Anisi, Fructus Foeniculi, Radix Althaeae, Fructus Carvi, Fructus Coriandri, Fructus Sabalae, Semen Erucae, Flores Sambuci and Herba Adonidis.

24. A method for inhibiting thrombin, wherein said method comprises the step of preparing a preparation having at least one unsaturated fatty acid, wherein said at least one unsaturated fatty acid has a carbon chain with a chain length of 18 or 20 carbon atoms, and wherein if said at least one unsaturated fatty acid has a chain length of 18 carbon atoms, said at least one unsaturated fatty acid contains 1 to 3 double bonds in said carbon chain, and if said at least one unsaturated fatty acid has a chain length of 20 carbon atoms, said at least one unsaturated fatty acid contains 1 to 4 double bonds in said carbon chain, and wherein the double bond or one of the double bonds is located at position 9 or 11 of the carbon chain, and wherein said at least one unsaturated fatty acid is present in an all-cis configuration.

25. The method according to claim 24, wherein said at least one unsaturated fatty acid is selected from the group consisting of oleic acid, linoleic acid, linolenic acid, eicosenoic acid, ricinoleic acid, vaccenic acid, eicosatrienoic acid and arachidonic acid.

26. A method for inhibiting thrombin, wherein said method comprises the step of preparing a preparation having at least one plant drug, wherein said at least one plant drug contains at least one unsaturated fatty acid or at least one triglyceride with said at least one unsaturated fatty acid, or with at least one of said at least one unsaturated fatty acid as fatty acid residue, said at least one unsaturated fatty acid having a carbon chain with a chain length of 18 or 20 carbon atoms and, in the case of a chain length of 18 carbon atoms, containing 1 to 3 double bonds in said carbon chain and, in the case of a chain length of 20 carbon atoms, containing 1 to 4 double bonds in said carbon chain, and wherein the double bond or one of the double bonds is located at position 9 or 11 of the carbon chain, and wherein said at least one unsaturated fatty acid is present in an all-cis configuration.

27. The method according to claim 26, wherein said at least one plant drug is selected from the group of plant drugs comprising Fructus Cardamomi, Fructus Sabalae, Fructus Anisi, Fructus Foeniculi, Radix Althaeae, Fructus Carvi, Fructus Coriandri, Fructus Sabalae, Semen Erucae, Flores Sambuci and Herba Adonidis.

28. A method for producing a preparation for inhibiting thrombin, wherein said method comprises the step of preparing said preparation by extracting one of dichloromethane or methanol from a plant drug to form a dichloromethane or methanol extract, adding a polyvinyl pyrrolidone solution to the dichloromethane or methanol extract and subsequent centrifugation to precipitate and withdraw a tanning agent contained in the dichloromethane or methanol extract, said preparation having at least one unsaturated fatty acid, wherein said at least one unsaturated fatty acid has a carbon chain with a chain length of 18 or 20 carbon atoms, and wherein if the unsaturated fatty acid has a chain length of 18 carbon atoms, said at least one unsaturated fatty acid contains 1 to 3 double bonds in said carbon chain, and wherein if said at least one unsaturated fatty acid has a chain length of 20 carbon atoms, said at least one unsaturated fatty acid contains 1 to 4 double bonds in said carbon chain, and wherein the double bond or one of the double bonds is located at position 9 or 11 of the carbon chain, and wherein the unsaturated fatty acid is present in an all-cis configuration.

29. The method according to claim 28, wherein said at least one unsaturated fatty acid is selected from the group consisting of oleic acid, linoleic acid, linolenic acid, eicosenoic acid, ricinoleic acid, vaccenic acid, eicosatrienoic acid and arachidonic acid.

30. The method according to claim 28, wherein the preparation is present in a form selected from the group consisting of a medicament, a food supplement, a dietetic salad oil, an additive for soups and an additive for sauces.

31. A method for producing a preparation for inhibiting thrombin, wherein said method comprises the step of preparing said preparation having at least one plant drug by extracting one of dichloromethane or methanol from said at least one plant drug to form a dichloromethane or methanol extract, adding a polyvinyl pyrrolidone solution to the dichloromethane or methanol extract and subsequent centrifugation to precipitate and withdraw a tanning agent contained in the dichloromethane or methanol extract, wherein said at least one plant drug contains at least one unsaturated fatty acid or at least one triglyceride with said at least one unsaturated fatty acid, or with at least one of said at least one unsaturated fatty acid as fatty acid residue, said at least one unsaturated fatty acid having a carbon chain with a chain length of 18 or 20 carbon atoms and, in the case of a chain length of 18 carbon atoms, containing 1 to 3 double bonds in said carbon chain and, in the case of a chain length of 20 carbon atoms, containing 1 to 4 double bonds in said carbon chain, and wherein the double bond or one of the double bonds is located at position 9 or 11 of the carbon chain, and wherein said at least one unsaturated fatty acid is present in an all-cis configuration.

32. The method according to claim 31, wherein said at least one plant drug is selected from the group of plant drugs consisting of Fructus Cardamomi, Fructus Sabalae, Fructus Anisi, Fructus Foeniculi, Radix Althaeae, Fructus Carvi, Fructus Coriandri, Fructus Sabalae, Semen Erucae, Flores Sambuci and Herba Adonidis.
